(19) United States
(12) Patent Application Publication
(10) Pub. No.: US 2024/0057944 A1
(43) Pub. Date: Feb. 22, 2024

(54) DEVICE AND METHOD OF CONTACTLESS PHYSIOLOGICAL MEASUREMENT WITH ERROR COMPENSATION FUNCTION

(71) Applicant: FaceHeart Inc., Hsinchu City (TW)

(72) Inventors: Yi-Chiao Wu, New Taipei City (TW); Li-Wen Chiu, Tainan City (TW); Meng-Liang Chung, Changhua County (TW); Bing-Fei Wu, Hsinchu City (TW)

(73) Assignee: FaceHeart Inc., Hsinchu City (TW)

(21) Appl. No.: 18/089,729

(22) Filed: Dec. 28, 2022

(30) Foreign Application Priority Data
Aug. 17, 2022 (TW) 111130978

(51) Int. Cl. A61B 5/00; A61B 5/024; G06V 40/16

(52) U.S. Cl. CPC A61B 5/7203; A61B 5/7267; A61B 5/02427; G06V 40/161

(57) ABSTRACT

A device of contactless physiological measurement with error compensation function is disclosed, and comprises a camera and a modular electronic device that is embedded with a face detection unit, a physiological parameter estimating unit, a feature extraction unit, an error compensation unit, and a physiological parameter generating unit therein. The face detection unit detects a face region from a user image, such that the physiological parameter estimating unit extracts an rPPG signal from the face region, so as to calculate a preliminary physiological parameter based on the rPPG signal. Simultaneously, the feature extraction unit extracts an error feature from the face region, such that the error compensation unit generates an error compensation parameter based on the error feature and the preliminary physiological parameter. Therefore, the physiological parameter generating unit conducts an addition operation of the error compensation parameter and the preliminary physiological parameter, thereby generating a physiological parameter.

### BACKGROUND OF THE INVENTION

### 1. Field of the Invention

The present invention relates to the technology field of contactless physiological measurement devices, and more particularly to a contactless physiological measurement device able to apply an error compensation to measured physiological parameters according to artifacts that are induced by motion and/or illumination variation.

### 2. Description of the Prior Art

Human face is an important information source for a human being, e.g., a man commonly looks washed out in case of having illness. Therefore, monitoring of physiological information is very important for assessing health and access to physiological data is not only necessary in clinical setting but it is becoming increasingly so also in other environments and applications related, for example, to telemedicine, personal fitness, e-commerce, trading and mental stress caused by the interaction with technology.

Accordingly, an optical measuring technique called photoplethysmography (PPG) is developed and therefore used to measure one people's physiological parameters, including pulse and heart rate (HR). With reference to FIG. 1, there is shown a schematic stereo diagram of a conventional contactless physiological measurement device using PPG technology. Moreover, FIG. 2 illustrates a block diagram of the conventional contactless physiological measurement device. As FIG. 1 and FIG. 2 shows, the conventional contactless physiological measurement device 1a principally comprises a camera 11a and an electronic device 12a coupled to the camera 11a, of which the electronic device 12a has a microprocessor 121a and a memory 122a coupled to the microprocessor 121a. Particularly, the memory 122a stores a face detection program 123a and a physiological parameters estimating program 124a. By such arrangements, in case of the face detection program 123a is executed, the microprocessor 121a is configured for determining a face region (i.e., ROI region) from an image that is acquired from a user by the camera 11a. Moreover, in case of the physiological parameters estimating program 124a is executed, the microprocessor 121a is configured for extracting a rPPG signal from the face region, thereby generating at least one physiological parameter like HR or pulse after applying at least one signal process to the rPPG signal.

Real experiences reveal that, artifacts induced by motion and/or illumination variation are found to affect the accuracy of the physiological parameters measured by the contactless physiological measurement device 1a. Accordingly, there are some anti-motion methods proposed for being applied in the contactless physiological measurement device 1a. In addition, at least one improved or advanced physiological parameters estimating program is developed for conducting the estimation of at least one physiological parameter with the reduction of the influence of the artifacts. However, it is a pity that, with the enhancement of the artifact influence, the proposed methods still fail to guarantee the accuracy of the physiological parameters measured by the contactless physiological measurement device 1a.

According to above descriptions, it is understood that there are still rooms for improvement in the conventional contactless physiological measurement device 1a including the face detection program 123a and the physiological parameters estimating program 124a. In view of this fact, inventors of the present application have made great efforts to make inventive research and eventually provided a device and method of contactless physiological measurement with error compensation function.

### SUMMARY OF THE INVENTION

The primary objective of the present invention is to disclose a device of contactless physiological measurement with error compensation function. The device comprises a camera and a modular electronic device, in which the modular electronic device includes a face detection unit, a physiological parameter estimating unit, a feature extraction unit, an error compensation unit, and a physiological parameter generating unit. The face detection unit detects a face region from a user image, such that the physiological parameter estimating unit extracts an rPPG signal from the face region, so as to calculate a preliminary physiological parameter based on the rPPG signal. Simultaneously, the feature extraction unit extracts an error feature from the face region, such that the error compensation unit generates an error compensation parameter based on the error feature and the preliminary physiological parameter. As a result, the physiological parameter generating unit conducts an addition operation of the error compensation parameter and the preliminary physiological parameter, thereby generating a physiological parameter.

In brief, this contactless physiological measurement device is configured so as to apply an error compensation to measured physiological parameters according to the artifacts that are induced by motion and/or illumination variation. In other words, despite the fact that the artifact influence is suddenly enhanced due to motion and/or illumination variation, this contactless physiological measurement device still can measure a user's physiological parameters with high accuracy.

For achieving the primary objective mentioned above, the present invention provides an embodiment of the device of contactless physiological measurement with error compensation function, comprising:
a camera, being disposed to face a user;
a modular electronic device, being coupled to the camera, and comprising a microprocessor and a memory, wherein the memory stores an application program, and the application program including instructions, such that in case the application program is executed, the microprocessor being configured for:
controlling the camera to acquire an image from the user;
detecting a face region from the image;
extracting an rPPG signal from the face region, so as to calculate a preliminary physiological parameter based on the rPPG signal;
extracting an error feature from the face region, so as to calculate an error compensation parameter based on the error feature and the preliminary physiological parameter; and
conducts an addition operation of the error compensation parameter and the preliminary physiological parameter, thereby generating a physiological parameter.

Moreover, the present invention also provides an embodiment of a method of contactless physiological measurement with error compensation function, which is compiled to be an application program so as to be stored in a memory of a modular electronic device, and is conducted by a microprocessor of the modular electronic device; the contactless physiological measurement method comprising steps of:
(1) controlling a camera that is coupled to the modular electronic device to photograph a user, so as to acquire an image;
(2) detecting a face region from the image;
(3) extracting an rPPG signal from the face region, and then calculating a preliminary physiological parameter based on the rPPG signal;
(4) extracting an error feature from the face region;
(5) calculating an error compensation parameter based on the error feature and the preliminary physiological parameter; and
(6) conducting an addition operation of the error compensation parameter and the preliminary physiological parameter, thereby generating a physiological parameter.

In one embodiment, the error feature comprises at least one that is selected from a group consisting of brightness, area of ROI region, area of skin region, signal-to-noise ratio (SNR), and two color difference components Cb and Cr.

In one embodiment, the error feature comprises frequency magnitude.

In one embodiment, the application program consists of a plurality of subprograms, and the plurality of subprograms comprises:
a first subprogram, being compiled to be integrated in the application program by one type of programming language, and including instructions for configuring the microprocessor to control the camera to acquire the image from the user;
a second subprogram, being compiled to be integrated in the application program by one type of programming language, and including instructions for configuring the microprocessor to apply a face detecting process to the image, so as to detect the face region from the image;
a third subprogram, being compiled to be integrated in the application program by one type of programming language, and including instructions for configuring the microprocessor to extract the rPPG signal from the face region, and then to calculate said preliminary physiological parameter based on the rPPG signal;
a fourth subprogram, being compiled to be integrated in the application program by one type of programming language, and including instructions for configuring the microprocessor to extract the error feature from the face region;
a fifth subprogram, being compiled to be integrated in the application program by one type of programming language, and including instructions for configuring the microprocessor to calculate said error compensation parameter based on the error feature and the preliminary physiological parameter; and
a sixth subprogram, being compiled to be integrated in the application program by one type of programming language, and including instructions for configuring the microprocessor to conduct an addition operation of the error compensation parameter and the preliminary physiological parameter, thereby generating a physiological parameter.

In one embodiment, the fifth subprogram includes a pre-trained error compensation parameter calculating model, such that in case the fifth subprogram is executed, the microprocessor is configured for calculating said error compensation parameter based on the error feature and the preliminary physiological parameter.

In one embodiment, wherein the plurality of subprograms further comprises:
a seventh subprogram, being compiled to be integrated in the application program by one type of programming language, such that in case the seventh subprogram is executed, the microprocessor being configured for applying a model training process to a machine learning model using a pre-collected training sample set, said error feature, said preliminary physiological parameter, and a reference physiological parameter corresponding to the preliminary physiological parameter, thereby producing said error compensation parameter calculating model.

In one embodiment, the physiological parameter comprises at least one selected from a group consisting of pulse, heart rate (HR), Heart rate variance (HRV), blood pressure, respiratory rate, and blood oxygen saturation (SpO₂).

In one embodiment, the camera and the modular electronic device are integrated in an electronic device, and the electronic device is selected from a group consisting of desktop computer, laptop computer, all-in-one computer, tablet computer, smart television, smart phone, and video door entry system.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a schematic stereo diagram of a conventional contactless physiological measurement device using PPG technology;

FIG. 2 shows a block diagram of the conventional contactless physiological measurement device;

FIG. 3 shows a schematic stereo diagram of a device of contactless physiological measurement with error compensation function according to the present invention

FIG. 4 shows a block diagram of the device of contactless physiological measurement with error compensation function according to the present invention;

FIG. 5A shows a data graph of an rPPG signal that is extracted from a face region of an imager acquired from a user;

FIG. 5B shows a data graph of a frequency-domain rPPG signal;

FIG. 5C shows a data graph of the frequency-domain rPPG signal that has received a filtering treatment; and

FIG. 6A and FIG. 6B show a flow chart of a method of contactless physiological measurement with error compensation function according to the present invention.

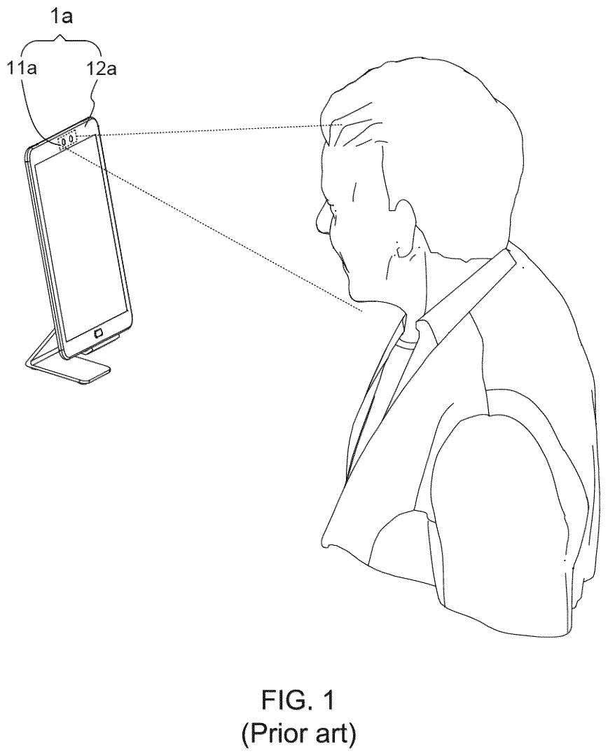

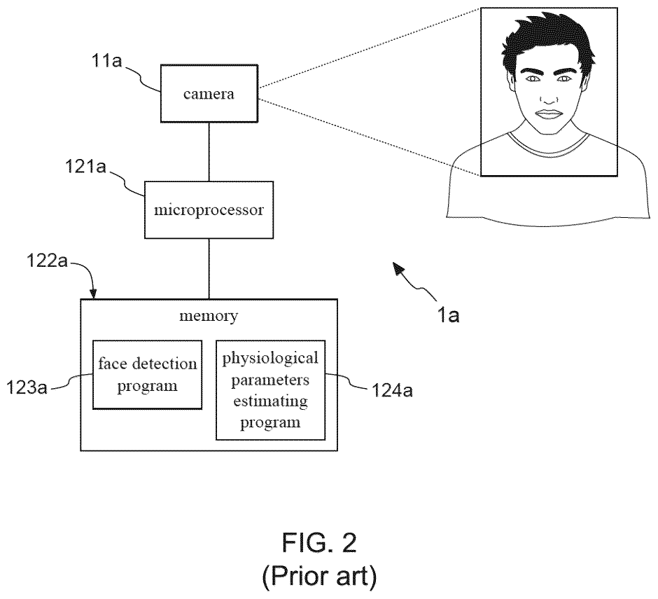

### DETAILED DESCRIPTION OF THE PREFERRED EMBODIMENTS

To more clearly describe a device and method of contactless physiological measurement with error compensation function according to the present invention, embodiments of the present invention will be described in detail with reference to the attached drawings hereinafter.

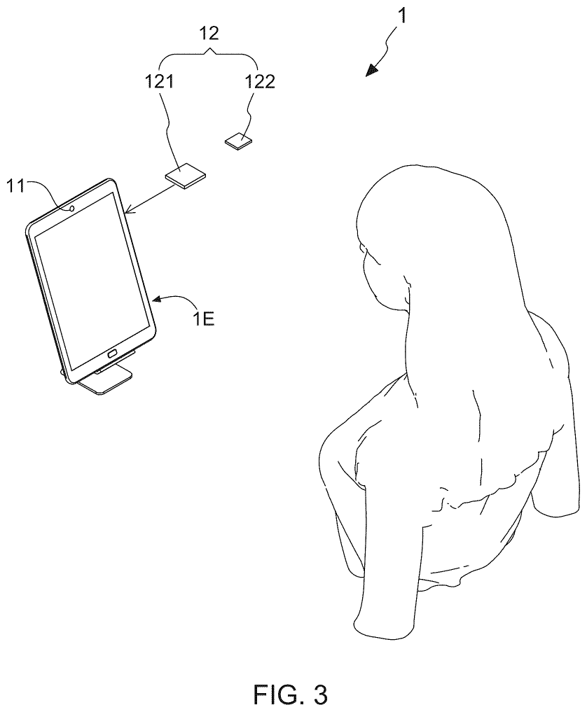

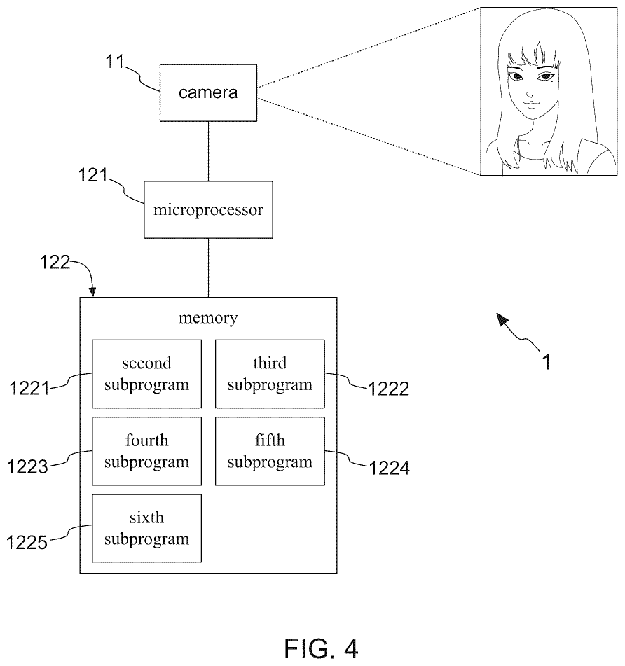

With reference to FIG. 3, there is shown a schematic stereo diagram of a device of contactless physiological measurement with error compensation function according to the present invention. Moreover, FIG. 4 illustrates a block diagram of the device of contactless physiological measurement with error compensation function according to the present invention. As FIG. 3 and FIG. 4 show, the present invention discloses a device 1 of contactless physiological measurement with error compensation function (“contactless physiological measurement device 1”, hereinafter), which principally comprises a camera 11 and a modular electronic device 12. In one practicable embodiment, the camera 11 and the modular electronic device 12 are integrated in an electronic device 1E, and the electronic device 1E can be a desktop computer, a laptop computer, an all-in-one computer, a tablet computer, a smart television, a smart phone, or a video door entry system. In another one practicable embodiment, the modular electronic device 12 is integrated in an electronic device 1E like a laptop computer, a desktop computer, or a tablet computer, and the camera 11 is coupled to the electronic device 1E, and the electronic device 1E.

In other practicable embodiment, the modular electronic device 12 is integrated in an electronic device 1E, and the electronic device 1E can be a cloud computing device or a server computer. In such case, it is able to constitute the contactless physiological measurement device 1 of the present invention by making the camera 11 be coupled to (communicated with) the electronic device 1E.

As FIG. 4 shows, the camera 11 is disposed to face a user, and the modular electronic device 12 is coupled to the camera 11. According to the present invention, the modular electronic device 12 comprises a microprocessor 121 and a memory 122, of which the memory 122 stores an application program, and the application program includes a plurality of subprograms. As FIG. 4 shows, the plurality of subprograms comprises a first subprogram, a second subprogram 1221, a third subprogram 1222, a fourth subprogram 1223, a fifth subprogram 1224, and a sixth subprogram 1225. As described in more detailed below, the first subprogram is compiled to be integrated in the application program by one type of programming language, and includes instructions for configuring the microprocessor 121 to control the camera 11 to acquire an image from a user.

As described in more detailed below, the second subprogram 1221 is compiled to be integrated in the application program by one type of programming language, and includes instructions for configuring the microprocessor 121 to apply a face detecting process to the image, so as to detect a face region (i.e., ROI region) from the image. In an exemplary embodiment, the second subprogram 1221 includes a pre-trained face detection model, wherein the pre-trained face detection model is produced after applying a model training process to a deep learning model like multi-task convolutional neural networks (MTCNN) model using a pre-collected training sample set.

On the other hand, the third subprogram 1222 is compiled to be integrated in the application program by one type of programming language, and including instructions for configuring the microprocessor 121 to extract the rPPG signal from the face region, and then to calculate said preliminary physiological parameter based on the rPPG signal. There is a need to explain that, the third subprogram 1222 includes a rPPG algorithm for use in the calculation of said preliminary physiological parameter. For example, rPPG algorithm is a CHROM algorithm proposed by literature document 1. Herein, literature document 1 is written by de Haan et. al, and is entitled with “Robust Pulse Rate From Chrominance-Based rPPG” so as to be published on IEEE Trans. Biomed. Eng., vol. 60(2013), no. 10.

As FIG. 4 shows, the fourth subprogram 1223 is compiled to be integrated in the application program by one type of programming language, and includes instructions for configuring the microprocessor 121 to extract an error feature from the face region. In one practicable embodiment, the error feature is a first feature F_{FQI} related to facial quality indices (FQIs), and comprises at least one of brightness, area of ROI region, area of skin region, signal-to-noise ratio (SNR), and two color difference components Cb and Cr. Moreover, in another one practicable embodiment, the the error feature is a second feature F_{MS} related to Frequency magnitude spectra (MS), and comprises frequency magnitude.

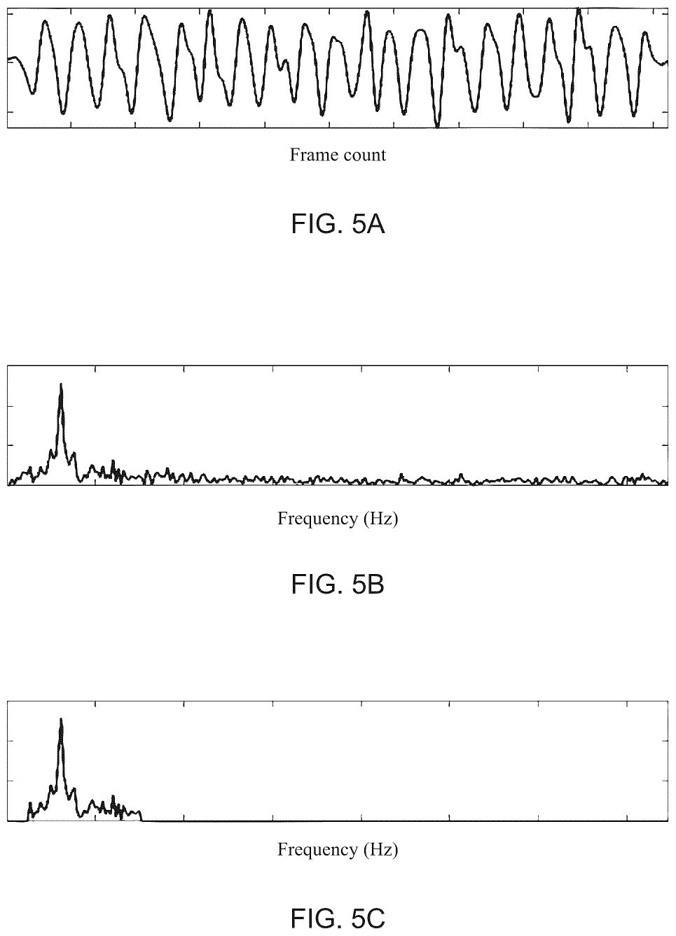

It is worth further explaining that, the first feature F_{FQI} can be directly extracted from the image acquired from the user. However, when conducting the extraction of the second feature F_{MS}, it needs to firstly extract an rPPG signal (as shown in FIG. 5A) from the face region, subsequently to apply a time domain-to-frequency domain converting process to the rPPG signal so as to obtain a frequency-domain rPPG signal (as shown in FIG. 5B), and consequently extracting the second feature F_{MS} from the frequency-domain rPPG signal (as shown in FIG. 5C) that has received a filtering treatment.

As FIG. 4 shows, the fifth subprogram 1224 is compiled to be integrated in the application program by one type of programming language, and includes instructions for configuring the microprocessor 121 to calculate an error compensation parameter based on the error feature and the preliminary physiological parameter. As described in more detailed below, the fifth subprogram 1224 includes a pre-trained error compensation parameter calculating model, such that in case the fifth subprogram 1224 is executed, the microprocessor 121 is configured for calculating said error compensation parameter based on the error feature and the preliminary physiological parameter. In one practicable embodiment, the plurality of subprograms further comprises a seventh subprogram, which is compiled to be integrated in the application program by one type of programming language, such that in case the seventh subprogram is executed, the microprocessor 121 is configured for applying a model training process to a machine learning model using a pre-collected training sample set, said error feature, said preliminary physiological parameter, and a reference physiological parameter corresponding to the preliminary physiological parameter, thereby producing said error compensation parameter calculating model. The machine learning model can be a stacked bidirectional long short-term memory model. In addition, said preliminary physiological parameter calculated by using CHROM algorithm is a first physiological parameter measured under the user is in a motion state or stays under an unstable ambient illumination. On the other hand, said reference physiological parameter calculated by using CHROM algorithm is a second physiological parameter measured under the user is in a stationary state or stays under an ambient illumination with slight or short-term disturbance (DIS).

Furthermore, the sixth subprogram 1225 is compiled to be integrated in the application program by one type of programming language, and includes instructions for configuring the microprocessor 121 to conduct an addition operation of the error compensation parameter and the preliminary physiological parameter, thereby generating a physiological parameter.

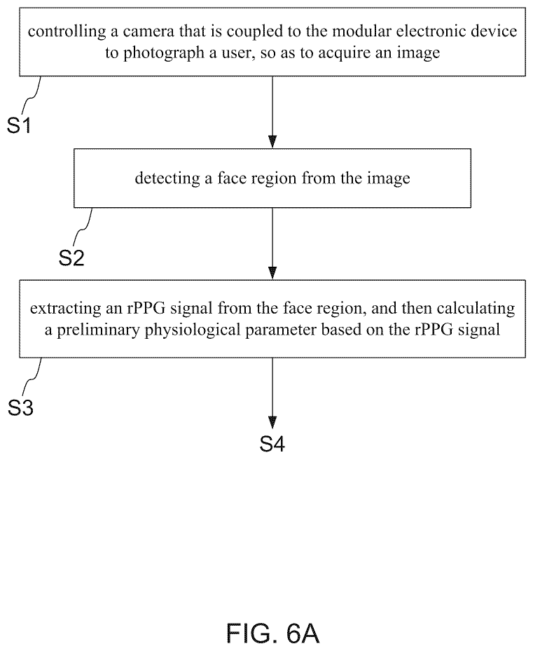

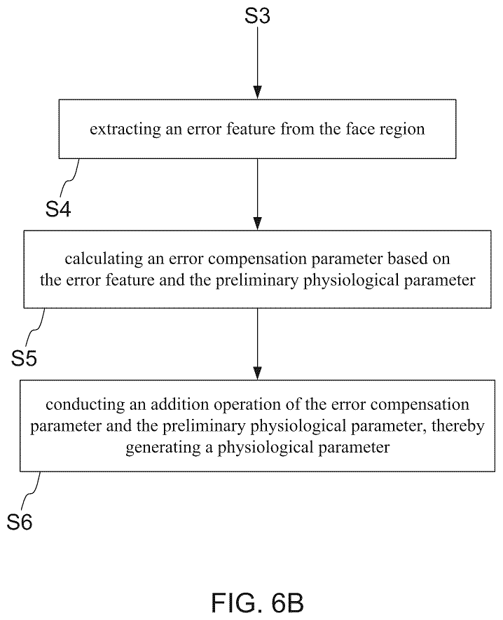

Moreover, the present invention also discloses a method of contactless physiological measurement with error compensation function. FIG. 6A and FIG. 6B show a flow chart of the method of contactless physiological measurement with error compensation function according to the present invention. As FIG. 4, FIG. 6A and FIG. 6B show, the method of contactless physiological measurement with error compensation function (“contactless physiological measurement method”, hereinafter) is being compiled to be an application program so as to be stored in a memory 122 of a modular electronic device 12, and being conducted by a microprocessor 121 of the modular electronic device 12. The method flow firstly proceeds to step S1. In step S1, the first subprogram is executed, such that the microprocessor 121 is configured to control the camera 11 to acquire an image from a user.

Subsequently, the method flow proceeds to step S2. In step S2, the second subprogram 1221 is executed, such that the microprocessor 121 is configured to control the camera 11 to apply a face detecting process to the image, so as to detect a face region from the image. Next, the method flow proceeds to step S3. In step S3, the third subprogram 1222 is executed, such that the microprocessor 121 is configured to extract an rPPG signal from the face region, and then to calculate a preliminary physiological parameter based on the rPPG signal. After that, the method flow proceeds to step S4. In step S4, the fourth subprogram 1223 is executed, such that the microprocessor 121 is configured to extract an error feature from the face region. Subsequently, the method flow proceeds to step S5. In step S5, the fifth subprogram 1224 is executed, such that the microprocessor 121 is configured to control the camera 11 to calculate an error compensation parameter based on the error feature and the preliminary physiological parameter. Consequently, the method flow proceeds to step S6. In step S6, the sixth subprogram 1225 is executed, such that the microprocessor 121 is configured to control the camera 11 to conduct an addition operation of the error compensation parameter and the preliminary physiological parameter, thereby generating a physiological parameter.

Therefore, through above descriptions, all embodiments and their constituting elements of the device and method of contactless physiological measurement with error compensation function according to the present invention have been introduced completely and clearly. Moreover, the above description is made on embodiments of the present invention. However, the embodiments are not intended to limit the scope of the present invention, and all equivalent implementations or alterations within the spirit of the present invention still fall within the scope of the present invention.

What is claimed is:

1. A contactless physiological measurement device, comprising:
a camera, being disposed to face a user;
a modular electronic device, being coupled to the camera, and comprising a microprocessor and a memory, wherein the memory stores an application program, and the application program including instructions, such that in case the application program is executed, the microprocessor being configured for:
controlling the camera to acquire an image from the user;
detecting a face region from the image;
extracting an rPPG signal from the face region, so as to calculate a preliminary physiological parameter based on the rPPG signal;
extracting an error feature from the face region, so as to calculate an error compensation parameter based on the error feature and the preliminary physiological parameter; and
conducts an addition operation of the error compensation parameter and the preliminary physiological parameter, thereby generating a physiological parameter.

2. The contactless physiological measurement device of claim 1, wherein the error feature comprises at least one that is selected from a group consisting of brightness, area of ROI region, area of skin region, signal-to-noise ratio (SNR), and two color difference components Cb and Cr.

3. The contactless physiological measurement device of claim 1, wherein the error feature comprises frequency magnitude.

4. The contactless physiological measurement device of claim 1, wherein the application program consists of a plurality of subprograms, and the plurality of subprograms comprising:
a first subprogram, being compiled to be integrated in the application program by one type of programming language, and including instructions for configuring the microprocessor to control the camera to acquire the image from the user;
a second subprogram, being compiled to be integrated in the application program by one type of programming language, and including instructions for configuring the microprocessor to apply a face detecting process to the image, so as to detect the face region from the image;
a third subprogram, being compiled to be integrated in the application program by one type of programming language, and including instructions for configuring the microprocessor to extract the rPPG signal from the face region, and then to calculate said preliminary physiological parameter based on the rPPG signal;
a fourth subprogram, being compiled to be integrated in the application program by one type of programming language, and including instructions for configuring the microprocessor to extract the error feature from the face region;
a fifth subprogram, being compiled to be integrated in the application program by one type of programming language, and including instructions for configuring the microprocessor to calculate said error compensation parameter based on the error feature and the preliminary physiological parameter; and
a sixth subprogram, being compiled to be integrated in the application program by one type of programming language, and including instructions for configuring the microprocessor to conduct an addition operation of the error compensation parameter and the preliminary physiological parameter, thereby generating a physiological parameter.

5. The contactless physiological measurement device of claim 4, wherein the fifth subprogram includes a pre-trained error compensation parameter calculating model, such that in case the fifth subprogram is executed, the microprocessor being configured for calculating said error compensation parameter based on the error feature and the preliminary physiological parameter.

6. The contactless physiological measurement device of claim 5, wherein the plurality of subprograms further comprises:
a seventh subprogram, being compiled to be integrated in the application program by one type of programming language, such that in case the seventh subprogram is executed, the microprocessor being configured for applying a model training process to a machine learning model using a pre-collected training sample set, said error feature, said preliminary physiological parameter, and a reference physiological parameter corresponding to the preliminary physiological parameter, thereby producing said error compensation parameter calculating model.

7. The contactless physiological measurement device of claim 1, wherein the physiological parameter comprises at least one selected from a group consisting of pulse, heart rate (HR), Heart rate variance (HRV), blood pressure, respiratory rate, and blood oxygen saturation (SpO₂).

8. The contactless physiological measurement device of claim 1, wherein the camera and the modular electronic device are integrated in an electronic device.

9. The contactless physiological measurement device of claim 1, wherein the modular electronic device is integrated in an electronic device, and the electronic device is selected from a group consisting of cloud computing device and server computer.

10. The contactless physiological measurement device of claim 8, wherein the electronic device is selected from a group consisting of desktop computer, laptop computer, all-in-one computer, tablet computer, smart television, smart phone, and video door entry system.

11. A contactless physiological measurement method, being compiled to be an application program so as to be stored in a memory of a modular electronic device, and being conducted by a microprocessor of the modular electronic device; the contactless physiological measurement method comprising steps of:
(1) controlling a camera that is coupled to the modular electronic device to photograph a user, so as to acquire an image;
(2) detecting a face region from the image;
(3) extracting an rPPG signal from the face region, and then calculating a preliminary physiological parameter based on the rPPG signal;
(4) extracting an error feature from the face region;
(5) calculating an error compensation parameter based on the error feature and the preliminary physiological parameter; and
(6) conducting an addition operation of the error compensation parameter and the preliminary physiological parameter, thereby generating a physiological parameter.

12. The contactless physiological measurement method of claim 11, wherein the error feature comprises at least one that is selected from a group consisting of brightness, area of ROI region, area of skin region, signal-to-noise ratio (SNR), and two color difference components Cb and Cr.

13. The contactless physiological measurement method of claim 11, wherein the error feature comprises frequency magnitude.

14. The contactless physiological measurement method of claim 11, wherein the application program consists of a plurality of subprograms, and the plurality of subprograms comprising:
a first subprogram, being compiled to be integrated in the application program by one type of programming language, and including instructions for configuring the microprocessor to control the camera to acquire the image from the user;
a second subprogram, being compiled to be integrated in the application program by one type of programming language, and including instructions for configuring the microprocessor to apply a face detecting process to the image, so as to detect the face region from the image;
a third subprogram, being compiled to be integrated in the application program by one type of programming language, and including instructions for configuring the microprocessor to extract the rPPG signal from the face region, and then to calculate said preliminary physiological parameter based on the rPPG signal;
a fourth subprogram, being compiled to be integrated in the application program by one type of programming language, and including instructions for configuring the microprocessor to extract the error feature from the face region;
a fifth subprogram, being compiled to be integrated in the application program by one type of programming language, and including instructions for configuring the microprocessor to calculate said error compensation parameter based on the error feature and the preliminary physiological parameter; and
a sixth subprogram, being compiled to be integrated in the application program by one type of programming language, and including instructions for configuring the microprocessor to conduct an addition operation of the error compensation parameter and the preliminary physiological parameter, thereby generating a physiological parameter.

15. The contactless physiological measurement method of claim 14, wherein the fifth subprogram includes a pre-trained error compensation parameter calculating model, such that in case the fifth subprogram is executed, the microprocessor being configured for calculating said error compensation parameter based on the error feature and the preliminary physiological parameter.

16. The contactless physiological measurement method of claim 15, wherein the plurality of subprograms further comprises:
a seventh subprogram, being compiled to be integrated in the application program by one type of programming language, such that in case the seventh subprogram is executed, the microprocessor being configured for applying a model training process to a machine learning model using a pre-collected training sample set, said error feature, said preliminary physiological parameter, and a reference physiological parameter corresponding to the preliminary physiological parameter, thereby producing said error compensation parameter calculating model.

17. The contactless physiological measurement method of claim 11, wherein the physiological parameter comprises at least one selected from a group consisting of pulse, heart rate (HR), Heart rate variance (HRV), blood pressure, respiratory rate, and blood oxygen saturation (SpO₂).

18. The contactless physiological measurement method of claim 11, wherein the camera and the modular electronic device are integrated in an electronic device.

19. The contactless physiological measurement method of claim 11, wherein the modular electronic device is integrated in an electronic device, and the electronic device is selected from a group consisting of cloud computing device and server computer.

20. The contactless physiological measurement device of claim 18, wherein the electronic device is selected from a group consisting of desktop computer, laptop computer, all-in-one computer, tablet computer, smart television, smart phone, and video door entry system.
